(19) United States
(12) Patent Application Publication
(10) Pub. No.: US 2023/0201776 A1
(43) Pub. Date: Jun. 29, 2023

(54) A GRAPHENE OXIDE-BASED MEMBRANE

(71) Applicants: Singapore University of Technology And Design, Singapore (SG); Singapore Health Services Pte Ltd, Singapore (SG)

(72) Inventors: Hui Ying YANG, Singapore (SG); Zhi Yi LEONG, Singapore (SG); Debajyoti Malakar ROY, Singapore (SG); Sreekanth KODURI, Singapore (SG)

(73) Assignees: Singapore University of Technology And Design, Singapore (SG); Singapore Health Services Pte Ltd., Singapore (SG)

(21) Appl. No.: 17/998,488

(22) PCT Filed: May 14, 2021
(86) PCT No.: PCT/SG2021/050266

§ 371 Date: Nov. 10, 2022

(30) Foreign Application Priority Data
May 14, 2020 (SG) 10202004499Q

(51) Int. Cl. B01D 71/02; C01B 32/198; C01B 32/194; A61M 1/16; B01D 69/10; B01D 67/00; B01D 69/12; B01D 61/42

(52) U.S. Cl. CPC B01D 71/0211; A61M 1/1696; B01D 61/428; B01D 67/00416; B01D 69/12; B01D 69/108; C01B 32/194; C01B 32/198; B01D 2323/30; C01B 2204/22; C01P 2002/72; C01P 2002/78

(57) ABSTRACT

A graphene oxide-based membrane There is provided a graphene oxide-based membrane comprising a substrate and a plurality of layers of single-layered graphene oxide formed on the substrate, each of the plurality of layers of single-layered graphene oxide is functionalised by at least one diamine functional group, wherein interlayer spacing between two adjacent layers of single-layered graphene oxide is ≤ 10 Å. The membrane may be comprised in an electrocapacitive unit. There is also provided a method of forming the membrane.

### TECHNICAL FIELD

The present invention relates to a graphene oxide-based membrane, and a method of forming the membrane.

### BACKGROUND

Dialysis is a common renal therapy recommended for patients with reduced or negligible kidney function. For dialysis, a dialysate is used to enable waste and toxins to diffuse from the patient’s blood across a membrane and into the dialysate. Once dialysis is completed, the spent dialysate is generally discarded as waste.

Sorbent systems have therefore been developed to reconstitute spent dialysates to minimise waste. These systems comprise sorbent layers in sorbent cartridges, the layers comprising zirconium phosphate, zirconium oxide, zirconium carbonate, aluminium oxide, and activated carbon, to remove uremic toxins, heavy metals and waste from spent dialysates. However, the drawback of such sorbent systems is that sorbent cartridges occupy a considerable amount of space and also are required to be replaced after each use in order to maintain high regeneration efficiency.

There is therefore a need for an improved system and method for reconstituting spent dialysate.

### SUMMARY OF THE INVENTION

The present invention seeks to address these problems, and/or to provide an improved membrane, particularly for use in a regenerative dialysis system.

According to a first aspect, the present invention provides a graphene oxide-based membrane comprising:
a substrate; and
a plurality of layers of single-layered graphene oxide formed on a surface of the substrate, each of the plurality of layers of single-layered graphene oxide is functionalised by at least one diamine functional group,
wherein interlayer spacing between two adjacent layers of single-layered graphene oxide is ≤ 10 Å.

In particular, the interlayer spacing between two adjacent layers of single-layered graphene oxide may be 5.5-7 Å.

The at least one diamine functional group may be comprised in a spacer molecule. The spacer molecule may comprise any suitable diamine functional group. For example, the spacer molecule may comprise, but is not limited to, linear diamines, aromatic diamines, or mixtures thereof. In particular, the linear diamine may be, but not limited to, methanediamine, ethylenediamine (EDA). The aromatic diamine may be, but not limited to, p-phenylenediamine.

According to a particular aspect, each of the plurality of layers of single-layered graphene oxide may be functionalised by at least one diamine functional group by cross-linking the at least one diamine functional group to the graphene oxide.

The substrate may be any suitable substrate. According to a particular aspect, the substrate may be a porous conductive substrate. In particular, the porous conductive substrate may be a porous carbon-based conductive substrate. For example, the porous conductive substrate may comprise a carbon fibre substrate.

According to a second aspect, the present invention provides a method of forming the graphene oxide-based membrane as described above, the method comprising:
dispersing graphene oxide flakes in a solvent to form a single-layered graphene oxide dispersion;
mixing the dispersion with spacer molecules comprising a diamine functional group to form a mixture;
passing the mixture through a surface of a substrate to form a nascent membrane on the surface of the substrate; and
heating the nascent membrane to form the graphene oxide-based membrane.

The dispersing may be by any suitable means. According to a particular aspect, the dispersing comprises ultrasonicating the dispersion.

The diamine functional group comprised in the spacer molecules may comprise any suitable diamine functional group. For example, the spacer molecular may comprise, a diamine functional group as described above in relation to the first aspect.

The passing may be by any suitable means. For example, the passing may comprise vacuum filtering the mixture through the surface of the substrate. The substrate may be any suitable substrate. In particular, the substrate may be as described above. According to a particular aspect, the substrate may be supported on a filter. The method may further comprise removing the filter after the passing.

According to a third aspect of the present invention, there is provided an electrocapacitive unit comprising the graphene oxide-based membrane according to the first aspect.

The electrocapacitive unit may further comprise an anode and a cathode such that the graphene oxide-based membrane may be between the anode and the cathode. The anode and the cathode may be of any suitable material. According to a particular aspect, each of the anode and the cathode may comprise porous carbon-based material.

The electrocapacitive unit may be comprised in a regenerative dialysis system for regenerating dialysate. The graphene oxide-based membrane comprised in the electrocapacitive unit may enable removal of excess ions from spent dialysate passing through the regenerative dialysis system.

The electrocapacitive unit may be comprised in an electrocapacitive module. The electrocapacitive module may comprise two or more electrocapacitive units arranged in a parallel configuration or a series configuration.

### BRIEF DESCRIPTION OF THE DRAWINGS

In order that the invention may be fully understood and readily put into practical effect there shall now be described by way of non-limitative example only exemplary embodiments, the description being with reference to the accompanying illustrative drawings. In the drawings:

FIG. 1 shows the X-ray diffraction (XRD) pattern of a graphene oxide-based membrane according to one embodiment of the present invention under wet and dry conditions;

FIG. 2 shows the XRD pattern of a graphene oxide membrane of prior art under wet and dry conditions;

FIG. 3 shows the interlayer spacing of a graphene oxide-based membrane according to one embodiment of the present invention and a graphene oxide membrane of prior art when immersed under different conditions;

FIG. 4 shows the X-ray photoelectron spectroscopy (XPS) spectra of a graphene oxide-based membrane according to one embodiment of the present invention as compared with a graphene oxide membrane of prior art;

FIG. 5 shows the XPS spectra of a graphene oxide membrane of prior art;

FIG. 6 shows the XPS spectra of graphene oxide-based membrane according to one embodiment of the present invention;

FIG. 7 shows a perspective view of an electrocapacitive unit according to one embodiment of the present invention;

FIG. 8 shows a schematic representation of a dialysis system comprising an electrocapacitive unit according to one embodiment of the present invention;

FIG. 9 shows a schematic representation of an electrocapacitive module comprising electrocapacitive units according to one embodiment of the present invention;

FIG. 10 shows the ion adsorption characteristics of an electrocapacitive unit comprising a membrane according to one embodiment of the present invention in 1 mmol/L monovalent and divalent cations;

FIG. 11 shows the ion adsorption characteristics of an electrocapacitive unit without a membrane in 1 mmol/L monovalent and divalent cations;

FIG. 12 shows the monovalent to divalent ion-selectivity of an electrocapacitive unit with and without a membrane according to one embodiment of the present invention;

FIG. 13 shows the ion-selectivity performance of an electrocapacitive unit comprising a membrane according to one embodiment of the present invention as a function of positive applied potential;

FIG. 14 shows the ion-selectivity performance of an electrocapacitive unit comprising a membrane according to one embodiment of the present invention as a function of negative applied potential; and

FIG. 15 shows the ion-selectivity performance of an electrocapacitive unit comprising a membrane according to one embodiment of the present invention under different operating modes.

### DETAILED DESCRIPTION

As explained above, there is a need for an improved system and method for reconstituting spent dialysate.

Generally, the method of the present invention relates to graphene oxide-based membranes and their integration in an electrocapacitive unit for the regeneration of spent dialysate. In particular, the membrane according to the present invention comprises ion-selective properties, thereby only removing excess ions from spent dialysate. For example, the membrane provides a barrier that creates asymmetric transport between the cations such that spent dialysates may be regenerated. The regenerated dialysate may therefore be suitable for use for at least a further round of dialysis without requiring any enrichment solution to reconstitute essential ions.

According to a first aspect, the present invention provides a graphene oxide-based membrane comprising:
a substrate; and
a plurality of layers of single-layered graphene oxide formed on a surface of the substrate, each of the plurality of layers of single-layered graphene oxide is functionalised by at least one diamine functional group,
wherein interlayer spacing between two adjacent layers of single-layered graphene oxide is ≤ 10 Å.

The graphene oxide-based membrane may be suitable for use in a regenerative dialysis system for regenerating spent dialysate. In particular, the graphene oxide-based membrane may be used in conjunction with an electrocapacitive method for removing excess ions from spent dialysate.

The at least one diamine functional group may be any suitable diamine functional group. According to a particular aspect, the diamine functional group may be comprised in a spacer molecule. The spacer molecule may comprise any suitable diamine functional group. For example, the spacer molecule may comprise, but is not limited to, linear diamines, aromatic diamines, or mixtures thereof. In particular, the linear diamine may be, but not limited to, methanediamine, ethylenediamine (EDA). The aromatic diamine may be, but not limited to, p-phenylenediamine.

Each of the plurality of layers of single-layered graphene oxide may be functionalised by the at least one diamine functional group by cross-linking the at least one diamine functional group to the graphene oxide. Accordingly, the graphene oxide-based membrane may comprise a plurality of layers of single-layered cross-linked graphene oxide, wherein the spacer molecule comprising the at least one diamine functional group is cross-linked to the graphene oxide.

The functionalisation of the single-layer graphene oxide layer enable the interlayer spacing between the plurality of layers to be controlled. Accordingly, by controlling the interlayer spacing between adjacent layers comprised in the membrane, the membrane may serve as a barrier to ions of a size larger than the interlayer spacing, thereby enabling ion-selective properties. For the purposes of the present invention, the term interlayer spacing may be defined as the perpendicular distance between two planes of graphene oxide layers. The interlayer spacing may be contrasted from d-spacing since d-spacing measures the perpendicular distance between two planes of graphene oxide layers as measured from the mid-points of the planes. Accordingly, in d-spacing, the size/thickness of a single plane of graphene oxide layer and the interlayer spacing is included.

According to a particular aspect, the interlayer spacing between two adjacent layers of single-layered graphene oxide may be 5.5-10 Å. For example, the interlayer spacing may be 5.8-9.5 Å, 6.0-9.3 Å, 6.2-9.0 Å, 6.5-8.5 Å,7.0-7.2 Å. In particular, the interlayer spacing between two adjacent layers of single-layered graphene oxide comprised in the membrane may be 5.5-7 Å. Even more in particular, the interlayer spacing may be 6-7 Å. Such an interlayer spacing may exclude or largely reduce the permeation of larger, divalent cations such as calcium and magnesium across the membrane. The movement of smaller, monovalent cations such as potassium and sodium may be penalized but at a smaller extent. The asymmetric transport of ions across the membrane may be effective at removing excess ions in spent (or waste) dialysate solution since spent dialysate solution may contain an excess of potassium followed by sodium ions. Although magnesium and calcium ions may also be present in excess, the excess concentrations are low and hence will be less affected.

The graphene oxide-based membrane may comprise a plurality of single-layered graphene oxide flakes. The single-layered graphene oxide flakes may generally form layers stacked on each other. According to a particular aspect, the membrane comprises 10²-10⁵ layers of single-layered graphene oxide flakes.

The membrane may have a suitable thickness. For example, the membrane may have a thickness of ≤ 5 µm. In particular, the membrane may have a thickness of 0.5-5 µm, 0.8-4.8 µm, 1.0-4.5 µm, 1.2-4.0 µm, 1.5-3.8 µm, 1.8-3.5 µm, 2.0-3.2 µm, 2.2-3.0 µm, 2.5-2.8 µm, 2.6-2.7 µm. Even more in particular, the thickness may be 1-2 µm.

The substrate comprised in the membrane may be any suitable substrate. The substrate may be any suitable substrate to provide mechanical strength to the membrane. In particular, the substrate may be any suitable substrate which is able to withstand convective forces when the membrane is in use in an electrocapacitive device for regenerating spent dialysate.

According to a particular aspect, the substrate may be a porous conductive substrate. In particular, the porous conductive substrate may be a porous carbon-based conductive substrate. For example, the porous conductive substrate may comprise a carbon fibre substrate.

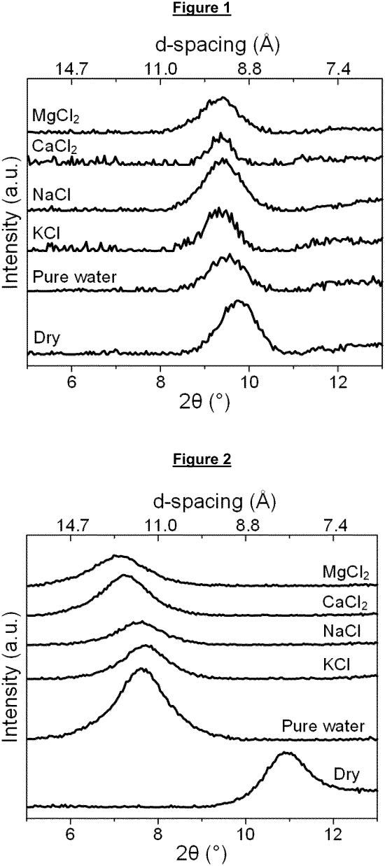

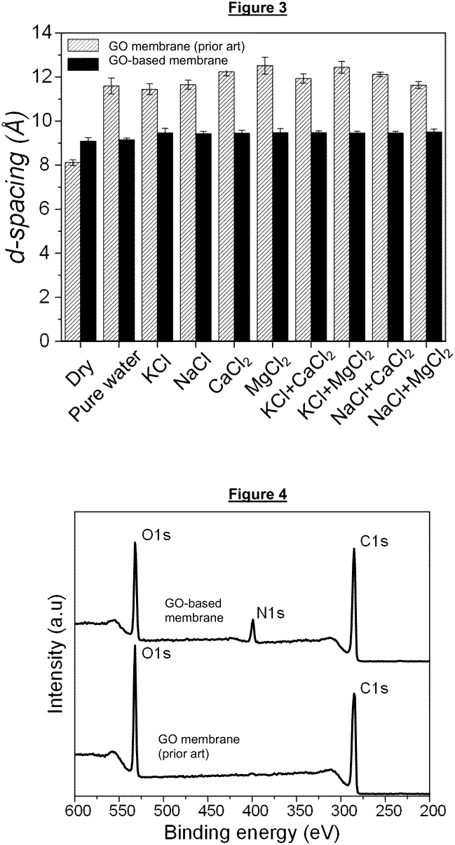

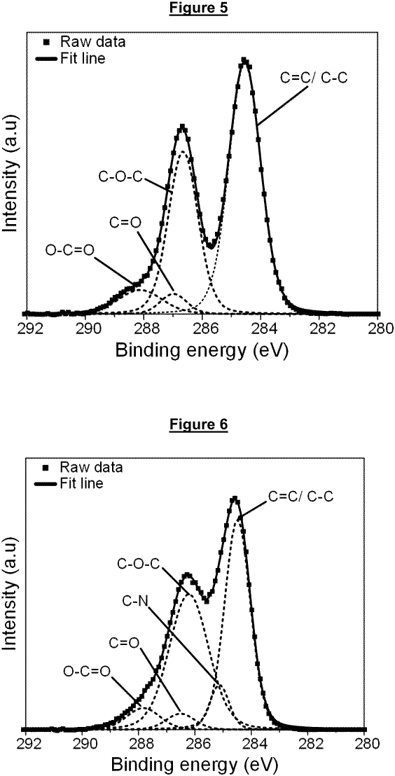

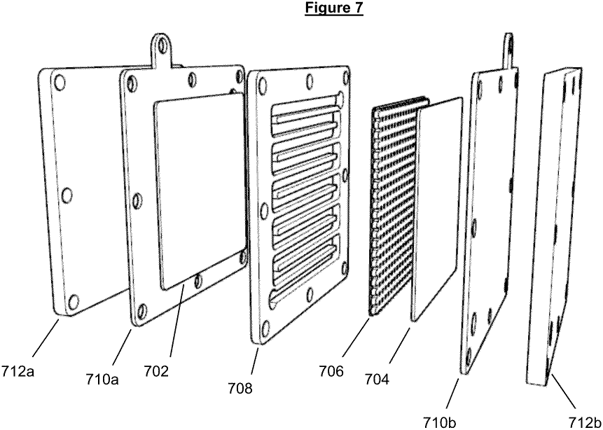

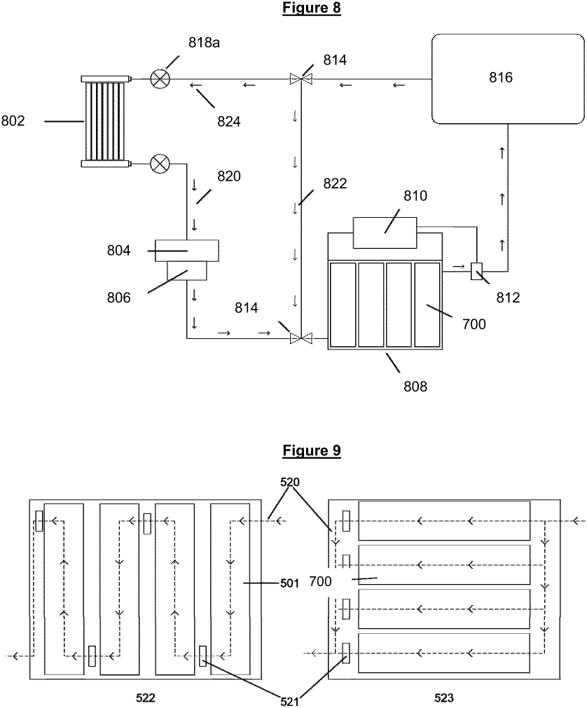

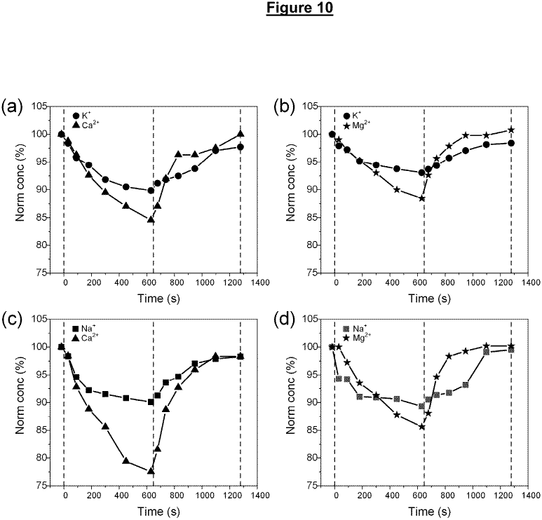

The X-ray diffraction (XRD) pattern of a graphene oxide-based membrane functionalised by EDA according to one embodiment of the present invention showing the changes in interlayer spacing when the membrane is dry, immersed in water and immersed in a variety of salt solutions is shown in FIG. 1. As seen from FIG. 1, when the membrane was immersed in water, the d-spacing of the adjacent layers comprised in the membrane increased from 9.15 ± 0.1 Å to 9.28 ± 0.2 Å due to intercalation of water molecules. After accounting for the size of a GO sheet (3.45 Å), the interlayer spacing between two adjacent layers may be about 5.83 Å, which is smaller than essential cations present in dialysate (e.g. K⁺ is 6.62 Å, Na⁺ is 7.16 Å, Ca²⁺ 8.24 Å and Mg²⁺ is 9.16 Å). In contrast, a typical graphene oxide membrane without functionalisation swelled from 8.11 ± 0.1 Å to 11.6 ± 0.4 Å in water, as shown in FIG. 2, which resulted in an interlayer spacing too large (8.15 Å) to function as an effective cut-off for ion exclusion.

It can also be seen from FIG. 1 that there was an invariance of peak position for the graphene oxide-based membrane according to the present invention, which indicates that the interlayer spacing did not change significantly when immersed in various salt solutions. On the other hand, a typical graphene oxide membrane showed clear peak shifts, as can be seen in FIG. 2, according to the type of salt solution it was immersed in. FIG. 3 provides the d-spacing values for both the membrane according to the present invention and a typical graphene oxide membrane based on the XRD pattern data.

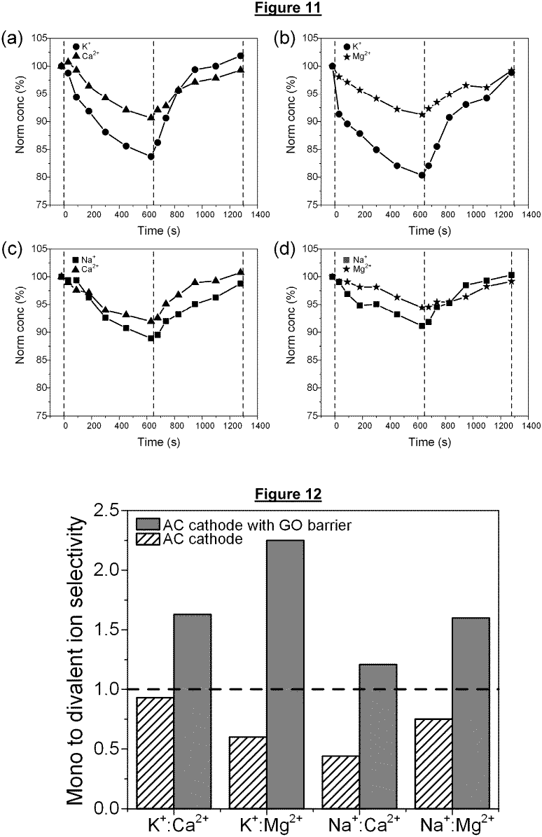

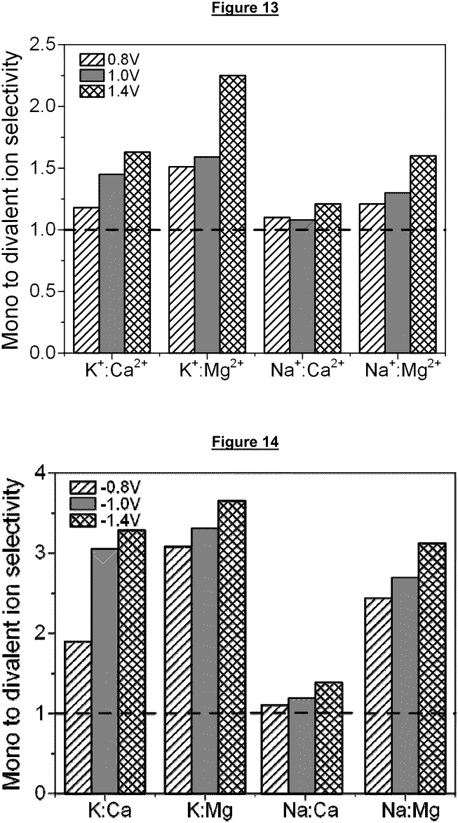

An elemental composition analysis was further performed using X-ray photoelectron spectroscopy (XPS) and the full spectra is presented in FIG. 4. FIG. 4 shows an obvious N1s peak attributed to aliphatic diamine in the membrane of the present invention. High resolution spectra of the C1s peak for a typical graphene oxide membrane, as shown in FIG. 5, showed four distinct peaks centered at 284.5 eV (C═C/C—C), 286.7 eV (C—O—C), 287.0 eV (C═O) and 288.1 eV (O—C═O), whereas a membrane according to the present invention showed five peaks at 284.5 eV (C═C/C—C), 285.1 eV (C—N), 286.2 eV (C—O—C), 286.5 eV (C═O) and 287.8 eV (O—C═O) (see FIG. 6). There were decreased peak intensities and lower binding energies for oxygen moieties such as C—O—C which indicated successful nucleophilic attacks by the diamine molecule on oxygen moieties. Overall, the atomic percentage of oxygen decreased from 26% to 21% while the nitrogen content increased from 0% to 7% which indicated a simultaneous amine functionalization and graphene oxide reduction (see Table 1).

TABLE 1

Atomic percentage of carbon, oxygen and nitrogen in membranes

| | Typical graphene oxide membrane (at %) | Graphene oxide-based membrane of present invention (at %) |
|---|---|---|
| C═C/C—C | 55.4 | 42.3 |
| C—O—C | 33.6 | 41.7 |
| C═O | 3.72 | 3.59 |
| O—C═O | 7.28 | 5.6 |
| C—N | - | 6.81 |

The amine functionalization had also altered the surface potential of the membrane according to the present invention to -18.6 mV, which was slightly higher than that of a typical graphene oxide membrane (-20.5 mV). Free diamine molecules are strongly positive due to protonation of the double amine groups but diamine molecules used in the fabrication of the membrane according to the present invention may be localised and bound between the graphene oxide layers due to formation of C—N bonds. Hence, there was not much change in the surface potential.

According to a second aspect, the present invention provides a method of forming the graphene oxide-based membrane as described above, the method comprising:
dispersing graphene oxide flakes in a solvent to form a single-layered graphene oxide dispersion;
mixing the dispersion with spacer molecules comprising a diamine functional group to form a mixture;
passing the mixture through a surface of a substrate to form a nascent membrane on the surface of the substrate; and
heating the nascent membrane to form the graphene oxide-based membrane.

The dispersing the graphene oxide flakes may be in any suitable solvent. For example, the solvent may be any suitable solvent in which the graphene oxide flakes and the subsequently added spacer molecule comprising the diamine functional group are soluble. According to a particular aspect, the solvent may be, but not limited to water, dimethylformamide (DMF), or mixtures thereof. In particular, the solvent may be water. Even more in particular, the water may be deionised water. When the solvent is any solvent other than water, the solvent may be diluted with water.

The dispersing may be by any suitable means. According to a particular aspect, the dispersing may comprise ultrasonicating the dispersion. In this way, the graphene oxide flakes may be uniformly dispersed in the water. The ultrasonicating may be under suitable conditions. For example, the ultrasonicating may be for a suitable period of time. In particular, the ultrasonicating may be for 15-60 minutes. The ultrasonication may be at a frequency of about 20-40 Hz. According to a particular aspect, the water in the ultrasonic bath may be maintained at a temperature of ≤ 25° C. In particular, the water in the ultrasonic bath may be maintained at a temperature of about 0-25° C., preferably closer to 0° C.

The dispersing may comprise dispersing 2-10 mg/mL graphene oxide flakes in about 5-40 mL of solvent. In particular, the dispersing may comprise dispersing 4-5 mg/mL graphene oxide flakes in about 10-20 mL of water. Increasing the thickness of the membrane yet to maintain a smooth and uniform morphology, a lower concentration of graphene oxide flakes at a higher volume of solvent may be used.

The mixing may be by any suitable means. For example, the mixing may comprise stirring the mixture.

The spacer molecules comprising the diamine functional group may be of a suitable size. For example, the spacer molecules may have an average particle size of ≤ 7 Å. In particular, the spacer molecule may have an average particle size of 1-7 Å, 2-6 Å, 3-5 Å, 4-4.5 Å. Even more in particular, the average particle size may be 6-7 Å.

The spacer molecule may be any suitable spacer molecule. For example, the spacer molecule may be a linear, rigid molecule. The spacer molecule may be, but not limited to, ethylenediamine. According to a particular aspect, the largest dimension for the spacer molecule may be the distance between two terminal amine groups.

The diamine functional group comprised in the spacer molecules may comprise any suitable diamine functional group. For example, the spacer molecular may comprise, a diamine functional group as described above.

The passing may be by any suitable means. The passing may be so as to enable formation of the graphene oxide-based membrane on the surface of the substrate. The substrate may be any suitable substrate. In particular, the substrate may be as described above in relation to the first aspect. According to a particular aspect, the substrate may be supported by a filter. In particular, the filter may have pores larger than the pores present in the substrate. For example, the filter may comprise, but is not limited to, nylon, cellulose, hydrophilic polytetrafluoroethylene (PTFE) or a combination thereof. The filter may also comprise any hydrophilic membrane.

According to a particular aspect, the passing may comprise vacuum filtering the mixture through the surface of the substrate. The vacuum filtering may be under suitable conditions. For example, the vacuum filtering may be at a vacuum pressure of about 0.01 MPa, particularly about 0.098 MPa, or at the maximum rated vacuum pressure.

The method may further comprise removing the filter after the passing such that the nascent graphene oxide-based membrane remains attached to the substrate. The removing may be by any suitable means.

The heating may comprise heating the nascent membrane at a suitable temperature. For example, the temperature may be 70-90° C. In particular, the temperature may be 72-88° C., 75-85° C., 77-83° C., 80-82° C. Even more in particular, the temperature may be 80-85° C. The heating may enable the spacer molecules to complete the cross-linking of the spacer molecules to the graphene oxide layers.

The method may further comprise washing the formed membrane. The washing may be using any suitable solvent. For example, the washing may be using any suitable alcohol. In particular, the alcohol may be, but not limited to, methanol or ethanol. Even more in particular, the alcohol may be methanol.

According to a third aspect of the present invention, there is provided an electrocapacitive unit comprising the graphene oxide-based membrane according to the first aspect. The electrocapacitive unit may be comprised in a regenerative dialysis system for regenerating dialysate. The graphene oxide-based membrane comprised in the electrocapacitive unit may enable removal of excess ions from spent dialysate passing through the regenerative dialysis system.

A non-limiting example of how the graphene oxide-based membrane according to one aspect of the present invention can be integrated into an electrocapacitive unit is shown in FIG. 7. The electrocapacitive unit 700 comprises two sections, an anode side and a cathode side. The anode 702 is defined as an electrode contacting the positive terminal of a power source whereas the cathode 704 is defined as an electrode contacting the negative terminal of the power source. The anode 702 and the cathode 704 may be formed of any suitable material, such as highly porous carbon-based materials such as, but not limited to, powdered activated carbon (AC), carbon nanotubes or mesoporous carbon. The membrane 706 may be placed such that the substrate surface opposite from the surface on which the graphene oxide sheets of the membrane 706 are formed faces the cathode 704 and the membrane surface comprising the graphene oxide sheets faces a spacer 708. The anode 702, cathode 704, membrane 706 and spacer 708 may be arranged between a pair of current collectors 710a and 710b. The various components of the electrocapacitive unit 700 may be encased between a casing 712a and 712b. The casing may be formed of a suitable material, such as, but not limited to, acrylic.

In use, spent dialysate enters through an inlet of the electrocapacitive unit 700, flows through a channel embedded within the spacer layer sandwiched by anode and cathode and exits through an outlet. The electrodes may be electrically charged by a power source which induces an electric field across the anode and cathode. Cations may be adsorbed by the cathode and anions may be adsorbed by the anode.

The electrocapacitive unit may be comprised in an electrocapacitive module. The electrocapacitive module may comprise two or more electrocapacitive units arranged in a parallel configuration or a series configuration.

An example of an electrocapacitive module incorporated in a regenerative dialysis system is as shown in FIG. 8. FIG. 8 shows spent dialysate 820 exit a dialyzer 802, and passes through an activated carbon layer 804 and urease layer 806 to remove heavy metals and urea before entering an electrocapacitive module 808. The electrocapacitive module 808 is connected to a microcontroller 810 which feeds information about the composition of dialysate exiting each electrocapacitive unit 700 comprised in the electrocapacitive module 808 to determine the charge mode and amount of charge dispensed to each electrocapacitive unit 700 comprised in the electrocapacitive module 808. The microcontroller 810 also takes input from a flow-sensor 812 at the exit of the electrocapacitive module 808 to control the dialysate flow.

If the dialysate is incompletely regenerated, a 3-way valve 814 allows the incomplete dialysate 822 to return to the electrocapacitive module 808 for another round of ion removal. Fully regenerated dialysate 824 is either stored in a dialysate bag 816 until use or directly used at the dialyzer 802. In particular, a pump 818a may be used for pumping the regenerated dialysate 824 and/or the spent dialysate 820 into and from the dialyzer 802.

FIG. 9 provides an example of an electrocapacitive module with four electrocapacitive units 700 in a series or parallel configuration. An electrocapacitive module is comprised of at least two units where one unit includes a membrane 706 and the other is without a membrane.

According to a fourth aspect, there is also provided a method of regenerating dialysate comprising removing excess ions from spent dialysate using the membrane according to the present invention. The composition of essential cations in a dialysate is typically: Na⁺ of 135-145 mmol/L, K⁺ of 1-2 mmol/L, Ca²⁺ of 1.25-1.5 mmol/L and Mg²⁺ of 0.25-0.5 mmol/L. Spent dialysate may contain an excess of these ions and the electrocapacitive module may be operated such that the excess ions are removed. Within the electrocapacitive module, the electrocapacitive units comprising the membrane may selectively adsorb monovalent cations such as Na⁺ and K⁺ over divalent cations, while the electrocapacitive units without the membrane may remove the divalent cations, such as Ca²⁺ and Mg²⁺. Selective adsorption of ions may be defined as the removal of a particular ion species without substantially affecting the concentration of other ions in the background. The selectivity may be tuned either by changing the spacer molecules and hence the formation of the membrane or by changing the electrode charging parameters. An ion-selective sensor 521 may be placed at the outlet of each electrocapacitive unit 700 to monitor the dialysate composition.

The electrocapacitive unit 700 may be operated either in a series configuration (522) to maximize rate of ion adsorption or a parallel configuration (523) to maximize the formation of a desired dialysate composition. In the series configuration, the input of the second electrocapacitive unit 700 follows the output of the first electrocapacitive unit 700 and so on. The concentrations of individual ions decrease after each electrocapacitive unit 700 and the composition of the solution changes between each electrocapacitive unit 700. In the parallel configuration, the inlet solution is the same for all electrocapacitive units 700 and the output of each electrocapacitive unit 700 is combined to produce an effluent solution of a common composition. The flow path of the dialysate is as shown by the dotted line 520.

FIGS. 10 and 11 illustrate normalized concentration adsorption curves of a electrocapacitive unit 700 with and without a membrane for one complete cycle, respectively. A potential of 1.4 V was applied for a period of about 10 mins to adsorb the ions and a short-circuit was applied after to discharge the electrodes. During discharging, ions were expelled from the electrodes and entered a waste stream. The discharging phase is also known as electrode regeneration and can occur after a set number of cycles or when ion adsorption has reached a minimum value. FIG. 10 clearly shows a selective adsorption of monovalent over divalent cations while FIG. 11 shows a higher divalent cation adsorption.

There is also provided a method to tune ion-selectivity by charge mode and amount of charge. FIG. 12 summarizes the monovalent to divalent ion-selectivity as determined in FIGS. 10 and 11. Under a constant potential mode, a cathode with a graphene oxide-based membrane according to the present invention may selectively adsorb monovalent over divalent cations. This ion-selectivity may be further enhanced by varying the applied potential as shown in FIG. 13. As shown in FIG. 13, as the applied potential increases, the ion-selectivity of monovalent to divalent ions increases.

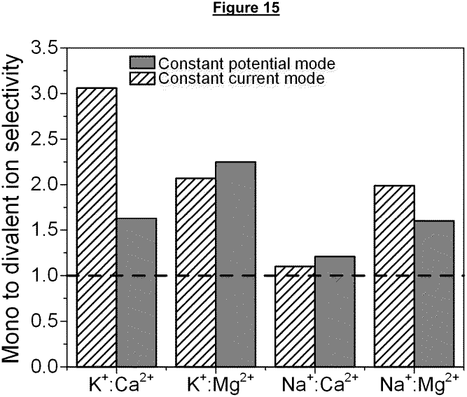

The ion-selectivity may also be enhanced further by applying a negative potential instead of a positive one. Even though the magnitude and direction of the electric field is the same, the polarity of the cathode is negative and that directly compels the movement of cations towards the cathode side. FIG. 14 shows enhancements in monovalent ion selectivity for all combinations of salt solutions except for the one with Na⁺ and Ca²⁺ which was approximately the same as when a positive potential was applied. However, a balance must be sought as a higher applied potential may result in a larger resistance drop across the electrodes and greater consumption of energy.

Another method to tune ion-selectivity is to charge the electrodes using constant current. A constant current mode typically results in a smaller resistance drop and produces a constant rate of ion adsorption which is desirable for a continuous regenerated stream of dialysate. FIG. 15 compares the performance of an electrocapacitive unit comprising a membrane according to the present invention under a constant potential mode versus a constant current mode. The constant current mode was charged to 1.4 V whereas the constant potential mode was set at 1.4 V. A higher ion-selectivity was observed for the constant current mode. By tuning the ion-selectivity in each electrocapacitive device, the output dialysate composition may therefore be controlled.

Whilst the foregoing description has described exemplary embodiments, it will be understood by those skilled in the technology concerned that many variations may be made without departing from the present invention.

Having now generally described the invention, the same will be more readily understood through reference to the following example which is provided by way of illustration, and is not intended to be limiting.

1. A graphene oxide-based membrane comprising:
a substrate; and
a plurality of layers of single-layered graphene oxide formed on a surface of the substrate, each of the plurality of layers of single-layered graphene oxide is functionalised by at least one diamine functional group,
wherein interlayer spacing between two adjacent layers of single-layered graphene oxide is ≤ ≤ 10 Å.

2. The membrane according to claim 1, wherein the interlayer spacing between two adjacent layers of single-layered graphene oxide is 5.5-7 Å.

3. The membrane according to claim 1, wherein the at least one diamine functional group is comprised in a spacer molecule.

4. (canceled)

5. The membrane according to claim 1, wherein the substrate is a porous conductive substrate.

6. The membrane according to claim 5, wherein the porous conductive substrate comprises a porous carbon-based conductive substrate.

7. The membrane according to claim 5, wherein the porous conductive substrate comprises a carbon fibre substrate.

8. The membrane according to claim 1, wherein each of the plurality of layers of single-layered graphene oxide is functionalised by at least one diamine functional group by cross-linking the at least one diamine functional group to the graphene oxide.

9. A method of forming a graphene oxide-based membrane according to claim 1, the method comprising:
dispersing graphene oxide flakes in a solvent to form a single-layered graphene oxide dispersion;
mixing the dispersion with spacer molecules comprising a diamine functional group to form a mixture;
passing the mixture through a surface of a substrate to form a nascent membrane on the surface of the substrate; and
heating the nascent membrane to form a graphene oxide-based membrane.

10. The method according to claim 9, wherein the dispersing comprises ultrasonicating the dispersion.

11. (canceled)

12. The method according to claim 9, wherein the passing comprises vacuum filtering the mixture through the surface of the substrate.

13. The method according to claim 9, wherein the substrate is supported on a filter.

14. The method according to claim 13, wherein the method further comprises removing the filter after the passing.

15. An electrocapacitive unit comprising a graphene oxide-based membrane according to claim 1.

16. The electrocapacitive unit according to claim 15, further comprising an anode and a cathode such that the graphene oxide-based membrane is between the anode and the cathode.

17. The electrocapacitive unit according to claim 16, wherein each of the anode and the cathode comprise porous carbon-based material.

18. The electrocapacitive unit according to claim 15, wherein the electrocapacitive unit is comprised in an electrocapacitive module.

19. The electrocapacitive unit according to claim 18, wherein the electrocapacitive module comprises two or more electrocapacitive units arranged in a parallel configuration.

20. The electrocapacitive unit according to claim 18, wherein the electrocapacitive module comprises two or more electrocapacitive units arranged in a series configuration.

21. The electrocapacitive unit according to claim 15, wherein the electrocapacitive unit is comprised in a regenerative dialysis system for regenerating dialysate.

22. The electrocapacitive unit according to claim 21, wherein the graphene oxide-based membrane enables removal of excess ions from spent dialysate passing through the regenerative dialysis system.
